(19) United States
(12) Patent Application Publication
(10) Pub. No.: US 2004/0118412 A1
(43) Pub. Date: Jun. 24, 2004

(54) Anatomical compression pad system

(76) Inventor: Gianbattista Alberto Piletti-Reyes, Guadalajara (MX)

Correspondence Address: KNOBBE MARTENS OLSON & BEAR LLP, 2040 MAIN STREET, FOURTEENTH FLOOR, IRVINE, CA, 92614, US

(21) Appl. No.: 10/329,662

(22) Filed: Dec. 24, 2002

(51) Int. Cl. A61F 5/37

(52) U.S. Cl. 128/876

(57) ABSTRACT

An anatomical compression system to facilitate a faster rehabilitation of a patient after a liposuction treatment or other treatment for enhancing the body shape. The anatomical system comprises a plurality of compression pads designed with a form-fitting shape for fitting perfectly predetermined body areas to apply properly compression forces to said body areas on which a liposuction treatment has been performed. The compression pads have one or more reinforcement sections specially disposed at specific locations to overlie body areas so as to cause compression to said body areas traumatized after a liposuction surgery. The objective of compressing the affected body areas is to minimize the amount of swelling following the liposuction surgery and therefore control the volume of the treated area to reach a desirable result.

### BACKGROUND OF THE INVENTION

1. Field of the Invention

The present invention relates to a compression pad system used inside a girdle for therapeutic use and more specifically to a novel pad system designed to compress or apply proper compression forces to the skin and underlying tissues of certain specific areas of the human body on which a liposuction or other treatment has been performed.

2. Prior Art

After a treatment of liposuction, body massage, mesotherapy, endermology has been carried out in the human body, different grades of inflammatory reaction and edema accumulation are produced. Particularly, when a liposuction treatment has been performed, wherein it implies to remove excess fat accumulated in certain parts of the body by inserting a suction cannula beneath the skin and removing said fat by the help of suction means connected to said cannula, it is thereafter necessary to compress those treated body areas (abdomen, legs, arms, hips, etc.) so as to minimize the amount of swelling following the treatment.

It is desirable to reduce at most the post-operative swelling of the tissue in order to promote a faster post-operative evolution of patient. At the present time, compression pads placed inside a girdle known under the trademarks EPIFOAM and TOPIFOAM are used, these pads being constructed from polyurethane foam coated by silicone. Such conventional pads are designed to cushion the treated area of patient body after a liposuction surgery, however, said pads disadvantageously consist in generally rectangular, flat sheets or foils having edges ending in a 90 angle configuration. The flat construction of prior art pads does not permit to be anatomically fitted to body areas treated by liposuction and therefore compression forces cannot be correctly applied thereon.

In addition, another problem with conventional compression pads is that wrinkles or creases on the patient skin are formed and a step shape between the compressed and non-compressed areas is produced because of the 90 angle configuration of their edges. Thus, the use of this type of compression bandages fails to provide the desired shrinkage of the body area traumatized by the therapeutic process. This is the main complain of the patient.

In view of the above, prior art pads are not the best choice for liposuction rehabilitation because the aim of applying properly compression forces to the treated body area to minimize the post-operative swelling is not fully accomplished.

Additionally, following a liposuction surgery performed in the abdominal or other area, permanent wrinkles or creases may be formed in this body portion because compression forces were not correctly applied to the patient skin and beneath tissues, thereby producing unsightly appearance. The same problem may occur in different body areas if proper compression forces are not applied to the tissue beneath which fat has been removed by a liposuction treatment.

### SUMMARY OF THE INVENTION

It is a first object of the invention to propose a pad compression system that fits anatomically the body of a patient who has been submitted to a therapeutic treatment (liposuction, mesotherapy, endermology, etc.) causing some inflammatory reaction and edema accumulation. The new pad system is formed to fit perfectly the body treated area such that compression forces be selectively applied thereon to minimize the swelling following one of the treatments mentioned above.

It is another object of the invention to provide a system of pads anatomically designed for different body areas on which a treatment has been carried out to mold the contour of the patient. The new system is intended to apply correctly compression forces to the treated areas of a patient without causing to develop wrinkles or creases on the patient skin. The compression pad comprises one or more reinforcement sections selectively placed therein.

Still another object of the invention is to mold the shape of the body in non surgical patients by applying compressive force and causing diminution of the fat tissue, that is the most susceptible for the compression, and it will cause atrophy of the said fat tissue in selective areas

Accordingly, the foregoing objects of the instant invention are accomplished by providing a novel compression pad system specially designed to conform each body area to be treated by a process like one of the described hereinabove. The novel compression pads are firstly adapted to be fitted properly to specific body areas and secondly they comprise at least one compression pad specially shaped in accordance with the affected body area so as to apply selective compression forces thereto for minimizing the tissue swelling following surgery.

Another advantage of the instant invention is that the edges of compression pads are angled and shaped with rounded ends to prevent the formation of edema imperfections in the patient skin.

Other objects and advantages of the invention will be apparent from the following description and accompanying drawings.

### BRIEF DESCRIPTION OF DRAWINGS

FIG. 1 is a frontal view of the compression pad for abdomen area depicted as worn by a female patient.

FIG. 2 is a perspective view depicting the back of patient to show the compression pad for the abdomen and also a compression pad for coccyx.

FIG. 3 is a perspective view of the compression pad designed to be placed on the abdomen area of a patient.

FIG. 4 is a frontal view of the abdomen compression pad.

FIG. 5 is a cross-sectional view of the abdomen compression pad.

FIG. 6 is a partial side view of the edge of compression pads having a rounded end portion

FIG. 7 is a frontal view of a compression pad placed on a patient to cover nape and superior part of the back

FIG. 8 is a frontal view of the compression pad shown in FIG. 7.

FIG. 9 is a cross-sectional view along line a-b of FIG. 8.

FIG. 10 is a perspective view of a compression pad for arm.

FIG. 11 is a frontal view of a compression pad for arm.

FIG. 12 is a cross-sectional view of the arm compression pad.

FIG. 13 is a perspective view of a compression pad for armpit.

FIG. 14 is a cross-sectional view along line x-x′ of FIG. 13.

FIG. 15 is a perspective view of a compression pad for hip.

FIG. 16 is a cross-sectional view along line y-y′ of FIG. 15.

FIG. 17 is a side view of a patient wearing a compression pad for the neck.

FIG. 18 is a frontal view of the neck compression pad.

FIG. 19 is a cross-sectional view along line z-z′ of FIG. 18 corresponding to the neck compression pad.

### DETAILED DESCRIPTION OF THE PREFERRED EMBODIMENTS OF THE INVENTION

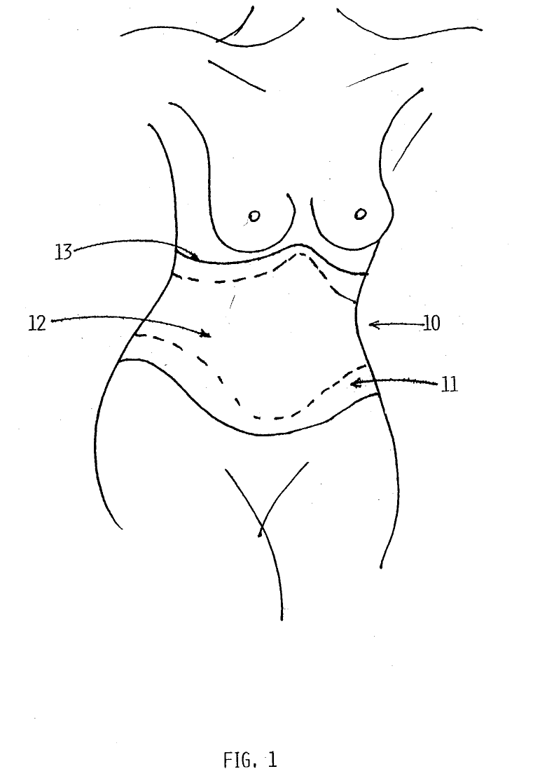

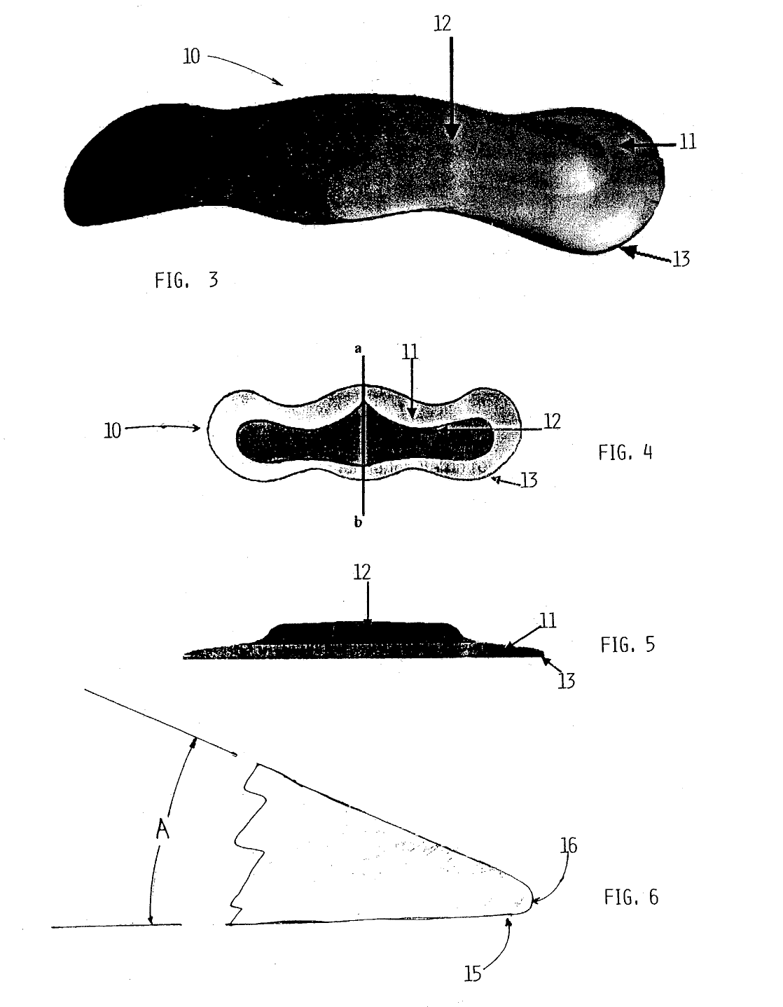

With reference to FIGS. 1-5, an anatomical compression pad (10) for the abdomen area is depicted therein, which pad is being worn (FIG. 1) by a woman after a therapeutic treatment, namely a liposuction process performed in the abdominal area. The compression pad (10) has a base element (11) made of polyurethane, silicone or a plastic material, the outer surface of pad being from about 1 to 3 inches thick depending upon the density of the polyurethane. The pad also includes reinforcement sections (12) comprising compression pads disposed interiorly and extending substantially through the central portion of the special shape of the abdominal pad (10). Reinforcement sections (12) are shaped in a manner as to apply selective compression forces not only to the abdominal area but also to the flanks (14) of the patient's waist in order to minimize the tissue swelling following a liposuction treatment and mold a harmonious figure. The thickness of said reinforcement sections (12) is preferably 30% to 40% greater than the thickness of the base element (11). An important excess fat is precisely accumulated in these body areas and therefore loose of skin results from a liposuction process. This looseness is highly avoided by using the instant compression pad.

As distinguished from a man compression pad, reinforcement sections (12) in the compression pad for a woman will be greater because it is desired to encompass at least partially the back region of the woman patient's waistline. As depicted in FIG. 1, reinforcement section (12) will be compressing the supraumbilical, infraumbilical, pubic, and waist flanks, the volume of said sections (12) being larger at the lowest region of the abdomen area. Contrarily, the compression pad in the man patient would lack of reinforcements in the flanks and said reinforcements will be located mainly in the middle of the abdomen, supra and infraumbilical.

As observed in FIG. 1, compression pad for abdomen area is designed to encompass a body area as follows:

From abdomen median line, compression pad (10) will extend about 2 centimeters from below the xyphoid appendix to the suprapubic line.

From clavicle median line, compression pad (10) will extend from rib edge line to the prolongation curve of the suprapubic line.

From anterior clavicle line, compression pad (10) will extend from the rib edge to the anterosuperior iliac spine. Because excess fat is usually accumulated below that body area in some patients, compression forces thereon will be needed therefore optional compression pads may be required.

From posterior clavicle line, compression pad (10) will extend about 3 to 6 centimeters from below the rib edge to about 2 to 4 centimeters adjacent the posterosuperior iliac spine.

From scapula median line, compression pad (10) will extend about 5 to 10 centimeters from below the scapula spine to approximately 3 to 6 centimeters from a line extending from the anterosuperior iliac spine to the beginning of the intergluteal fold.

In accordance with the above teachings, the body portions being covered by the abdomen compression pad (10) will be receiving selective compression forces. Naturally, a higher compression will be applied to the body areas overlaid by the reinforcement sections (12).

As depicted in FIG. 6, the outer edges (15) of each compression pad embodiment described herein are designed to form an end portion (16) having an angle A between 20° and 45° so as to diminish gradually the pressure at said end portions coming into contact with the patient skin. Thus, the outermost section of the end portion (16) is rounded-shaped to avoid possible breakage of the pad material and to prevent the formation of edema defects in the patient skin.

The technical characteristics of the different compression pad embodiments allow for applying selective pressure against the body area previously treated. For example, higher pressure will be effected by the reinforcement sections, while less pressure will be performed at the base element region and almost no pressure will be applied to at the edge portions of the compression pads.

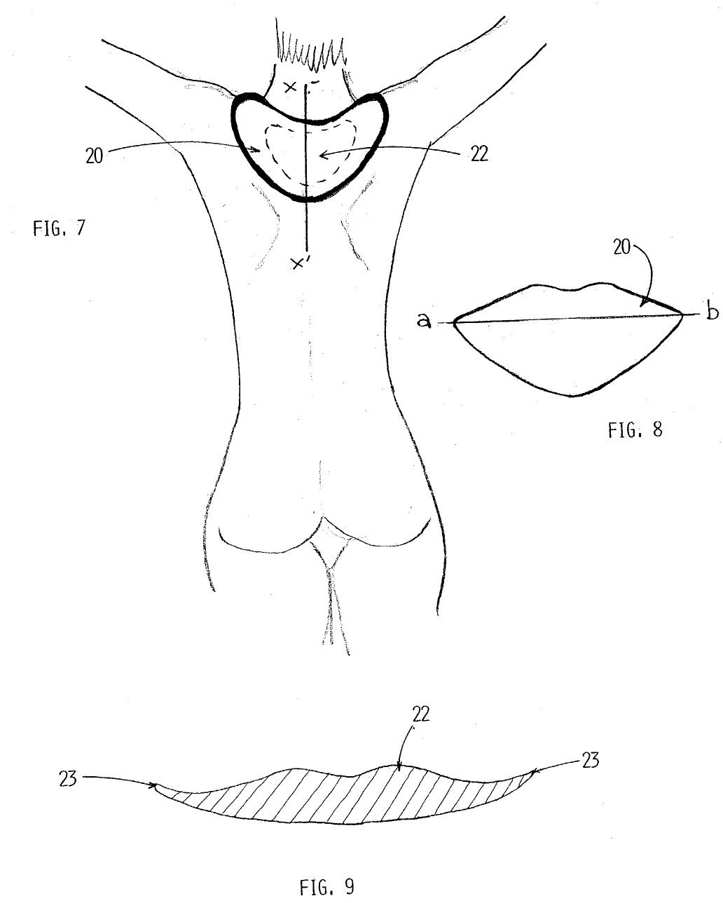

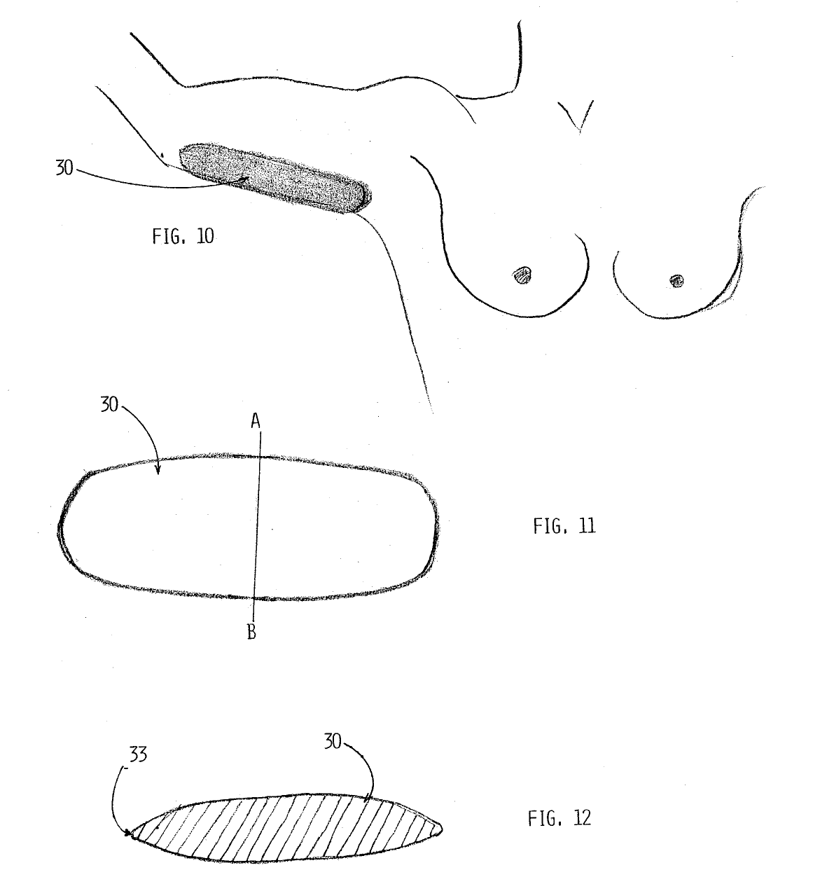

Now referring to FIG. 7, it depicts a compression pad (20) specially designed for the nape and the superior portion of the back. Once placed, this pad will extend downwardly approximately 15 to 20 centimeters (dependant upon the size) from the bony apophysis of the eighth cervical vertebra, and it will extend laterally approximately 12 to 15 centimeters from the median line x-x′. The nape and back compression pad (20) includes compression reinforcement sections (22) located at the valleys, beside the bony apophysis, which are intended to be compressed after a liposuction procedure. Similarly, the thickness of the nape pad will decrease from the reinforcement sections (22) to the outer edges (23) of said pad.

With reference to FIG. 11, a compression pad (30) for arm is depicted therein. As distinguished from the nape and abdomen pads, pad (30) has no reinforcement section and it merely consists in a oval shape member (30). However, the outer edges (33) of pad (30) are also rounded as proposed for the other compression pads.

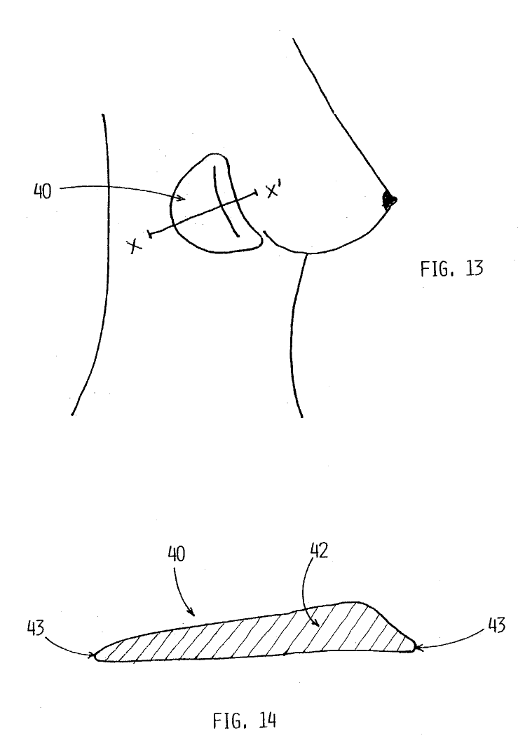

Referring now to FIGS. 13 and 14, a compression pad (40) for armpit is shown therein, said pad also including a reinforcement section (42) directed to enhance the breast of a woman patient and the pectoral for a man patient. As observed in FIG. 14, outer edges (43) of this armpit pad (40) are conveniently rounded.

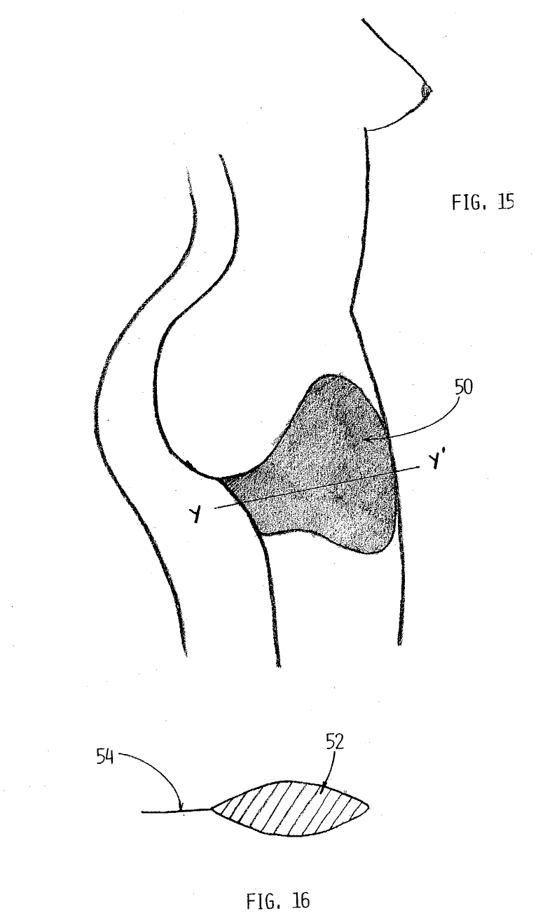

A compression pad (50) for hip is depicted in FIG. 15, said pad being placed on a woman patient. Pad (50) includes also a reinforcement section (52) and it is shaped to compress properly the hip region. Besides, compression pad (50) has an extension (54) to overlay the lower gluteal area.

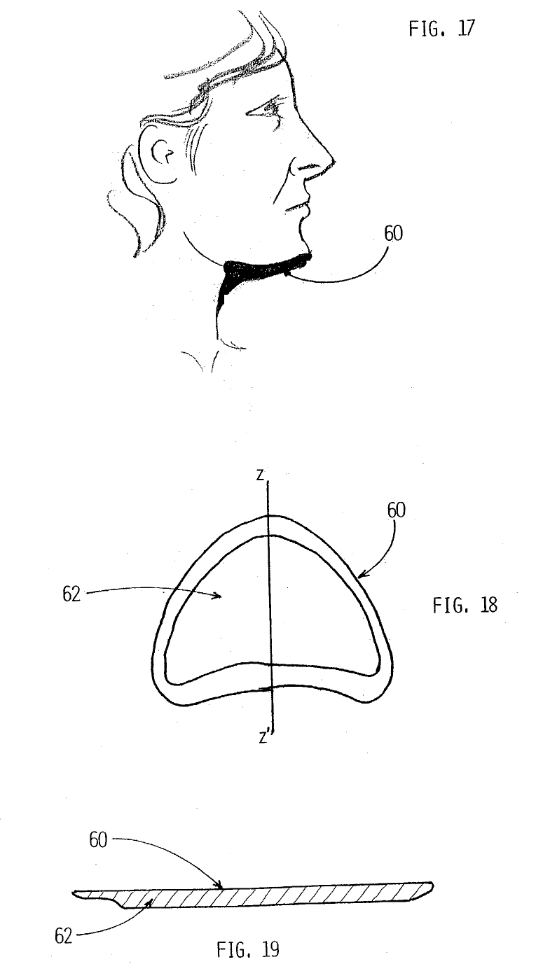

FIGS. 17-19 are related to a compression pad (60) for the neck. The pad (60) is designed also to include a reinforcement section (62) to apply appropriate compression to the area treated by a therapeutic process.

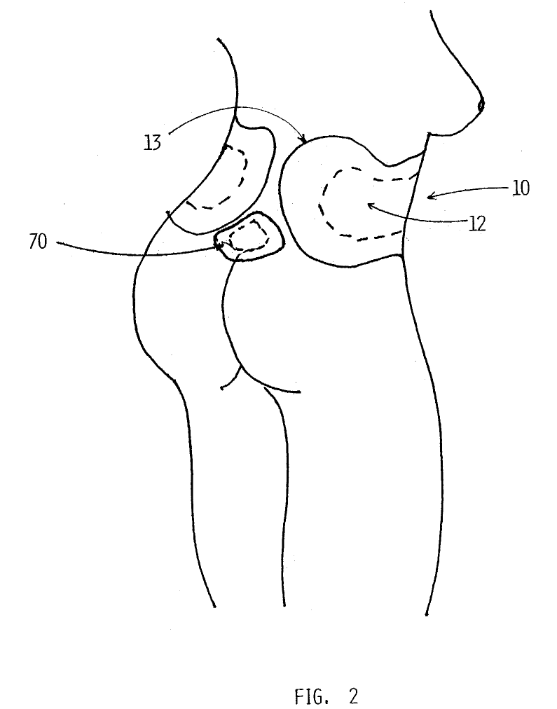

Finally, a compression pad (70) for coccyx is shown in FIG. 2. As observed, this pad is rhombus shape and its central portion is thicker than the outer edges because of the reinforcement section of said pad (70).

According to one embodiment of the invention, the base element and the reinforcement section for each of compression pads described herein may be made of the same material, the density of them being identical or different. In addition, if compression pads are made of polyurethane, it may optionally include a fine embossing cover of silicone disposed thereon.

As disclosed hereinbefore, the instant compression system provides compression pads specially designed to be adjusted anatomically to different body areas, namely, abdomen and flanks, back, arms, hips, thighs, armpit, neck, external pectoral region, crotch, nape, buttock, knees, coccyx, etc. Each pad will be of a form-fitting feature to compress those areas desired to be shaped and the size and form of the compression pads will be dependant upon the size and sex of the patient.

All of the compression pads described hereinabove will be kept on the patient's body area by a conventional girdle.

Having described the presently preferred embodiments, in accordance with the present invention, it is believed that other modifications, variations and changes will be suggested to those skilled in the art in view of the teachings set forth herein. It is, therefore, to be understood that all such variations, modifications, and the changes are believed to fall within the scope of the present invention as defined by the appended claims.

1. Anatomical compression pad system to shape the corporal contour, said system comprising a plurality of compression pads having a form-fitting shape so as to apply correctly compression forces to predetermined body areas of a patient following a liposuction or other treatment; each form-fitting compression pad comprises a base element, and at least one reinforcement section conveniently disposed at selected locations to overlie specific body areas affected by a liposuction treatment.

2. The anatomical compression pad system of claim 1, wherein outer edges of each compression pad are shaped in a manner as to form a rounded end portion.

3. The anatomical compression pad system of claim 1, wherein outer edges have an angular plane from 20° to 45°.

4. The anatomical compression pad system of claim 1, wherein the compression pad is designed specially to the shape of the patient's body area.

5. The anatomical compression pad system of claim 1, wherein the compression pad are so shaped to prevent as much as possible the amount of swelling resulting from a liposuction surgery.

6. The anatomical compression pad system of claim 1, wherein the reinforcement sections are adapted to apply compression forces to the skin and beneath tissues of a patient following a liposuction surgery or other treatment.

7. The anatomical compression pad system of claim 1, wherein the base element and the reinforcement section have identical density.

8. The anatomical compression pad system of claim 1, wherein said base element and said reinforcement section have different densities.

9. The anatomical compression pad system of claim 1, wherein said compression pads are specially designed at least for neck, arms, nape, back, armpit, abdomen, flanks, coccyx, hips, crotch, thighs, and knees.

10. The anatomical compression pad system of claim 1, further including a silicone cover on the surface of said pad.

11. The anatomical compression pad system of claim 1, wherein the thickness of compression pads decreases from the reinforcement sections to the outer edges thereof.

12. The anatomical compression pad system of claim 1, wherein the thickness of the reinforcement section is 30% to 40% greater than the thickness of the base element.
